(19) United States
(12) Patent Application Publication
(10) Pub. No.: US 2009/0117188 A1
(43) Pub. Date: May 7, 2009

(54) Methods of Augmenting or Repairing Soft Tissue

(76) Inventors: Julia B. Gershkovich, Lexington, MA (US); Peter K. Jarrett, Sudbury, MA (US); Kevin C. Skinner, Andover, MA (US); Gary D. Monheit, Birmingham, AL (US)

Correspondence Address: GENZYME CORPORATION;LEGAL DEPARTMENT, 15 PLEASANT ST CONNECTOR, FRAMINGHAM, MA, 01701-9322, US

(21) Appl. No.: 12/262,563

(22) Filed: Oct. 31, 2008

Related U.S. Application Data
(60) Provisional application No. 60/984,823, filed on Nov. 2, 2007.

(51) Int. Cl. A61K 9/14; A61K 31/08; A61P 43/00

(52) U.S. Cl. 424/484; 514/723

(57) ABSTRACT

Methods of repairing or augmenting soft tissue in a subject are described. The methods include injecting into a subject composition comprising a biodegradable, polymerizable macromer, the macromer comprising a water soluble polymer modified with one or more biodegradable moieties; and polymerizing the macromer to provide a hydrogel, thus repairing or augmenting the soft tissue.

### RELATED APPLICATIONS

This application claims the benefit of U.S. provisional application Ser. No. 60/984,823 filed Nov. 2, 2007. The entire teaching is incorporated herein by reference.

### TECHNICAL FIELD

This invention relates to methods of repairing or augmenting soft tissue.

### BACKGROUND

The repair or augmentation of soft tissue defects or contour abnormalities caused by facial defects, acne, surgical scarring or aging has proven to be very difficult. A number of materials have been used to correct soft tissue defects with varying degrees of success. In the past, small amounts of liquid silicone were used to correct minor soft tissue defects where minimal mechanical stress was present at the recipient site. Reconstituted injectable bovine collagen has also been used as a treatment for soft tissue defects. However, safety measures must be employed with this material to avoid allergic reactions to the bovine proteins in the collagen. Injectable implants of biocompatible ceramic particles in aqueous gels were first proposed by Wallace et al. in U.S. Pat. No. 5,204,382. The implants consisted of ceramic particles of calcium phosphate from a nonbiological source, mixed with an aqueous gel carrier in a viscous polymer (such as polyethylene glycol, hyaluronic acid (e.g., cross-linked hyaluronic acid containing compositions), poly(hydroxyethyl methacrylate) and collagen). Although these materials are generally nontoxic, nonabsorbable particulate materials in the formulation could lead to the migration of these particles.

Thermoplastic and thermosetting defect fillers were originally described by Dunn et al. in U.S. Pat. Nos. 4,938,763, 5,278,201 and 5,278,202. In these patents, Dunn proposes the use of both a thermoplastic material with a solvent and a thermosetting material with a curing agent to form solid implants in situ. Although the biodegradable materials Dunn suggests for use as thermoplastics appear acceptable, the solvents necessary to dissolve them for injection into tissue appear to be less than acceptable. Additionally, Dunn's thermoplastic and thermosetting materials have limited utility in filling soft tissue because they form more rigid solids. Similar commercially available materials exhibit ultimate yield stresses of approximately 10,000 psi; in comparison, human skin exhibits ultimate yield stresses of from 500 to 2,000 psi.

Current dermal fillers on the market including hyaluronic acid derived (such as Restylane, Juvederm, Prevelle) or collagen (Zyplast, Zyderm) are particulate and biodegradable and do not offer long lasting effects.

New soft tissue augmentation materials need to be developed. Ideally, any new augmentation material would have several important characteristics. For example, any new augmentation material could be completely bioabsorbable to avoid the possibility of long term chronic irritation of tissues or migration of nonabsorbable materials over time to different areas of the body. The new augmentation materials could also provide soft tissue augmentation for a sufficient amount of time, thus avoiding frequent readministration of the augmentation material. Furthermore, new soft tissue augmentation materials could be easy to administer preferably by injection. Finally, the ideal soft tissue augmentation material would have the appropriate degree cohesiveness and pliability for the tissue into which the new material is being implanted to provide life like, natural looking tissue augmentation.

### SUMMARY

Therefore, it is an object of the present invention to provide a safe, injectable, long lasting, cohesive, bioabsorbable material for soft tissue repair and augmentation.

Biodegradable, polymerizable macromers such as those macromers in FocalGel material can be used to repair and/or augment soft tissue. The macromers can be administered to a subject, for example, by injection intradermally or subdermally, and once administered, polymerized in the subject to provide a hydrogel, thereby repairing or augmenting the soft tissue of the subject. Upon administration, the material molded prior to polymerization to provide a cosmetically acceptable result and polymerized. The resulting hydrogel can provide a safe and effective means of repairing and/or augmenting soft tissue, for example, repairing soft tissue abnormalities due diseases such as lipoatrophy found in AIDS patients.

In one aspect, the invention features a method of repairing or augmenting soft tissue in a subject, the method comprising

a. injecting into a subject in need thereof a composition comprising a biodegradable, polymerizable macromer, the macromer comprising a water soluble polymer modified with one or more biodegradable moieties; and

b. polymerizing the macromer to provide a hydrogel wherein the hydrogel to soft tissue have a normalized compliance ratio of from about 0.05 to about 3, thus repairing or augmenting the soft tissue.

In some embodiments, the compliance ratio is from about 0.1 to about 2.0 relative to the soft tissue, for example, from about 0.1 to about 1.0 relative to the soft tissue.

In some embodiments, the macromer is polymerized by irradiating through the skin of the subject with visible light.

In some embodiments, the subject is irradiated with visible light for from about 10 seconds to about 120 seconds, for example, the subject is irradiated with visible light for at least about 30 seconds, or at least about 40 seconds.

In some embodiments, the macromer is polymerized by irradiating the subject with blue-green light. In some embodiments, the macromer is polymerized by irradiating the subject with thermal energy.

In some embodiments, the water soluble polymer is PEG, for example, the PEG has a molecular weight of from about 10,000 to about 35,000 Daltons.

In some embodiments, the water soluble polymer is a block-copolymer, for example, the block-copolymer is an ethyleneoxide and propyleneoxide.

In some embodiments, the macromer is biodegradable. In some embodiments, the macromer comprises a plurality of hydrolysable linkages. In some embodiments, the hydrolyzable linkages are selected from the group consisting of esters or carbonates.

In some embodiments, the water soluble polymer is modified with an acrylate-capped poly (L-lactide). In some embodiments, the water soluble polymer is PEG.

In some embodiments, the water soluble polymer is modified with a poly (trimethylene carbonate). In some embodiments, the water soluble polymer is PEG.

In some embodiments, the water soluble polymer is modified with an poly (L-lactide) and poly (trimethylene carbonate) and an acrylate endcap. In some embodiments, the water soluble polymer is PEG.

In some embodiments, the composition farther comprises a photo-initiator, for example, a dye such as eosin.

In some embodiments, the composition further comprises a rheology modifier, for example, hyaluronic acid (HA) or carboxymethyl cellulose (CMC).

In some embodiments, the composition is substantially free of organic solvent.

In some embodiments, the composition further comprises a drug such as an non-steroidal anti-inflammatory, an analgesic, a vitamin such as E, C, A, D or K, an anti-oxidant, an alpha hydroxyl acid such as lactic acid or a polymer capable of releasing such drug, vitamin, anti oxidant or alpha-hydroxyacid or any combination thereof.

In some embodiments, the hydrogel has a strain or elongation before fracture substantially similar to the expected strain during normal use of the soft tissue to which it augments or repairs.

In some embodiments, the hydrogel has a strain or elongation before fracture greater than the expected strain during normal use of the soft tissue to which it augments or repairs.

In some embodiments, the hydrogel has a reversible elongation at least about 150% as great as an expected strain of the soft tissue which is augments or repairs.

In some embodiments, the hydrogel has an elastic modulus which is less than about 150 kPa.

In some embodiments, the hydrogel has an ultimate yield stress of from about 500 to about 2,000 psi.

In some embodiments, the macromer is injected subdermally.

In some embodiments, the macromer is polymerized by irradiating least a part of the skin of the subject. In some embodiments, the skin is irradiated for at least about 30 seconds.

In some embodiments, the macromer is injected intradermally. In some embodiments, the macromer is polymerized by irradiating at least a part of the skin of the subject. In some embodiments, the skin is irradiated for at least about 30 seconds.

In some embodiments, the method also includes shaping the macromer. In some embodiments, the macromer is shaped during polymerization of the macromer. In some embodiments, the macromer is polymerized by irradiating through the skin of the subject.

In some embodiments, the method also includes repeating steps a) and b) at least one time, e.g., at least two times.

In some embodiments, the subject is a mammal, e.g., a human.

In some embodiments, the method includes repairing facial tissue, for example, decreasing the appearance of at least one facial line, wrinkle, crease, or fold.

In some embodiments, the method includes augmenting breast, lip, cheek, chin, forehead, buttocks, hand, neck or earlobe tissue in a subject. In some embodiments, the method includes decreasing the appearance of a dermal dimple, e.g., a dimple component of a scar.

In some embodiments, the composition is administered with a red tinted syringe.

In some embodiments, the soft tissue remains substantially augmented or repaired for at least about 1 month, e.g., at least about 2 months or at least about 6 months.

In some embodiments, the hydrogel elicits a mild fibrotic response in the subject.

In some embodiments, the composition comprises a two part system, and wherein the polymerization is initiated via a redox system.

In some embodiments, the polymerization occurs over a period of from about 30 seconds to about 2 minutes.

### DETAILED DESCRIPTION

As used herein, a “biocompatible” material is one that stimulates only a mild, often transient, implantation response, as opposed to a severe or escalating response. Biocompatibility may be determined by histological examination of the implant site at various times after implantation. One sign of poor biocompatibility can be a severe, chronic, unresolved phagocytic response at the site. Another sign of poor biocompatibility can be necrosis or regression of tissue at the site.

As used herein, a “biodegradable” material is one that decomposes under normal in vivo physiological conditions into components that can be metabolized or excreted. Functional groups having degradable linkages are incorporated into the structure of the hydrogel matrix to provide for its resorption over time. These functional groups may be incorporated within the macromers to form part of the backbone of the polymer strands of the hydrogel or as crosslinks between the polymer strands. Examples of degradable units may include, but are not limited to, esters, carbonates, and the like. In some embodiments, a hydrogel described herein fully degrades after about 3 months, after about 6 months, after about 1 year, or after about 2 years.

The properties of the hydrogels disclosed herein are referred to as “materials properties”, and include:

the “Young's modulus” (of elasticity) which is the limiting modulus of elasticity extrapolated to zero strain;

the “elastic modulus” which is any modulus of elasticity, not limited to Young's modulus, and may include “secant modulus” and other descriptors of non-linear regions of the stress-strain curve;

the “bulk” or “compressive” modulus which is used in its usual sense of ratio of stress to a designated compressive strain;

the “elongation at failure” which is the relative strain or extension of a test specimen at which any irreversible or hysteresis-inducing change occurs in the specimen; and

the “elongation at break” or “elongation at rupture” which is the relative strain (extension) of a test specimen at which mechanical rupture occurs.

The term “compliance” as used herein is used in a general sense, and refers for example to the ability of an implant to closely match the physiological and mechanical properties of tissues at the implant site, except when “compliance” is used in a specific technical sense as the reciprocal of a modulus.

As applied to a relatively thin, flat material such as a tissue, “normalized compliance” (NC) is defined herein as the strain, (i.e., the elongation or compression per unit length of a specimen), divided by the applied force per unit cross-sectional area, further divided by the thickness of the specimen. Hence, for a sample having a width, w, (for example, the width of the clamps of the testing apparatus), and a thickness, t, when an applied force, F, produces a strain, S, then the compliance, C, is

$$C = \frac{S}{F / wt} = \frac{S \cdot wt}{F}$$

and the normalized compliance is

$$NC = \frac{C}{t} = \frac{S}{F / w} = \frac{Sw}{F}$$

i.e., the strain in the sample divided by the force per unit width applied to the sample. The normalized compliance allows direct comparison of the forces required to deform the tissue versus a coating on the tissue (e.g., a hydrogel described herein), without regard to the relative thicknesses of these materials.

The normalized compliance ratio (abbreviated NCR) is defined as the value of the normalized compliance of the tissue or other substrate divided by the normalized compliance of the hydrogel. When both measurements are conducted on strips of the same width and at the same force, the NCR is simply the ratio of the strains at a particular force. A low NCR (less than 1) is obtained when the hydrogel is easier to deform than the tissue, while a high NCR (greater than 1) is obtained when the tissue is easier to deform than the hydrogel.

As used herein, the term “elastomer” refers to a polymeric material which at room temperature is capable of repeatedly recovering in size and shape after removal of a deforming force. In some embodiments, an elastomer is a material which can be repeatedly stretched to twice its original length and will repeatedly return to its approximate length on release of the stress.

The phrase “elastomeric materials” is a phrase which has been used in the literature. There are many publications describing structure-property relationships of elastomers and other deformable materials. Lower elastic modulus and, frequently, an increased reversible elongation to break or fracture, are found when any of the following occur:

1. The distance between nodes or junctions or more crystalline (“hard”) segments increases.

2. The crosslink density decreases. This may be controlled by amount of crosslinker, nature of crosslinker, and degree of cure, as well as by segment length of either the crosslinked species or the crosslinking species, where different.

3. For a material at equilibrium with a continuous phase, an increase in the plasticization of the elastomer by the continuous phase. For applications wherein the continuous phase is water, more particularly physiological saline, increasing hydrophilicity tends to increase compliance.

The term “mild fibrotic response,” when used herein means a response causing production, deposition, and/or contraction of extracellular matrix within the subject resulting from the injection and/or deposition of a composition or hydrogel described herein, which does not result in excessive inflammation and/or irritation. The mild fibrotic response results in some matrix deposition and fibrogenesis in the subject at the sight of the injection and can prolong the effects of the injection in the subject.

The details of one or more embodiments of the invention are set forth in the accompanying drawings and the description below. Other features, objects, and advantages of the invention will be apparent from the description and drawings, and from the claims.

### Macromer Containing Compositions and Hydrogels

The compositions described herein provide a biocompatible, polymeric hydrogel. The hydrogel is biodegradable, and generally is eliminated by the subject within about up to five years.

### Compositions Forming a Hydrogel Matrix

To achieve the above properties, the hydrogel is formed primarily of in-situ polymerized macromers, the macromers being themselves polymers or copolymers of one or more monomers having reactive groups providing resorbable linkages and polymerizable sites for biodegradability and polymerization. The macromers have sufficient hydrophilic character to form water-absorbent polymerized gel structures, and are at least dispersible in a substantially aqueous solution, and preferably are water-soluble. In some preferred embodiments, the compositions comprising the macromers are substantially free of organic solvent.

The macromers are preferably generally made predominantly of synthetic materials to provide hydrogels that are preferably highly compliant with soft tissue and/or connective tissue. The hydrogels are preferably covalently crosslinked in-situ to ensure that they are retained at the site of application until the hydrogels degrade within the subject and are eliminated.

### Monomer and Macromer Components of the Hydrogel

Monomers and macromers which are suitable for forming hydrogels (“referred to here in this section collectively as “monomers”) have one or more of the following properties: water solubility, partially macromeric in character, containing hydrophilic groups, and being covalently reactive. When crosslinked to form gels, the resulting gels are generally, elastic, and compliant.

The monomers are preferably water soluble. Water soluble materials are soluble to at least about 0.1 gram per liter of a substantially aqueous solvent. A substantially solvent comprises at least about 50% by weight of water, and less than about 50% by weight of a non-aqueous, water-miscible solvent. If the polymers are not entirely water soluble, they are generally dispersible in water, and form micelles, typically with the aid of non-aqueous, water-miscible solvents. The non-aqueous solvent is generally present in an amount that does not damage the tissue. Thus only a small amount of non-aqueous, water-miscible solvent should be present in the pre-gelled composition to minimize tissue irritation. Up to about 10% by weight of the solution can be a non-aqueous, water-miscible solvent (e.g., less than about 9%, less than about 8%, less than about 7%, less than about 6%, less than about 5%, less than about 4%, less than about 3%, less than about 2%, less than about 1%). In some preferred embodiments, the compositions described herein are substantially free of organic solvent. Examples of non-aqueous, water-miscible solvents include ethanol, isopropanol, N-methylpyrrolidone, propylene glycol, glycerol, low molecular weight polyethylene glycol, DMSO, Benzyl alcohol, and benzyl benzoate. Liquid surfactants, such as poloxamers (e.g., PLURONIC™ surfactants) and some polyethylene glycol derivatives (e.g., some TWEEN™ surfactants) can also be used as non-aqueous, water-miscible solvents.

The monomers are preferably at least partially macromeric (e.g., when injected, for example, as a blend), and are more preferably substantially to completely macromeric. Macromers tend to be innocuous to tissue because they will not readily diffuse into or penetrate cells. A macromer is a reactive monomer consisting of a polymeric material with a number-average or weight-average molecular weight of about 500 Daltons or more and at least one reactive group. To form a crosslinked gel by chain-growth polymerization, the macromers, along with any other smaller monomers, in a solution must contain on average more than one reactive group (which may be a covalently reactive group or a group that binds non-covalently to other macromers). For polymerizations involving step-growth polymerization, the macromers must contain on average more than two reactive groups, and the solution typically contain approximately equal numbers of the two different types of reactive groups. An example of step-growth polymerization is gelation by formation of urethane linkages from the reaction of isocyanate with hydroxyl groups. For free-radical polymerization of unsaturated materials (chain-growth polymerization), the monomers must contain on average more than one reactive group to crosslink.

The macromers generally have significant hydrophilic character so as to form water-absorbent gel structures. At least some of the macromers, and preferably most of the macromers, contain hydrophilic domains. A hydrophilic domain in a macromer is a hydrophilic group, block, or region of the macromer that would be water soluble if prepared as an independent molecule rather than being incorporated into the macromer. Hydrophilic groups are required for water dispersibility or solubility, and for retention of water by the gel after gelation, or upon rehydration after drying. The hydrophilic groups of the macromers are preferably made predominantly or entirely of synthetic materials. Synthetic materials of controlled composition and linkages are typically preferred over natural materials due to more consistent degradation and release properties. Examples of useful synthetic materials include those prepared from poly(ethylene oxide) (i.e., PEG), partially or fully hydrolyzed poly(vinyl alcohol), poly(vinylpyrrolidone), poly(ethyloxazoline), poly(ethylene oxide)-co-poly(propylene oxide) block copolymers (e.g., Pluronics™) (poloxamers and meroxapols), and poloxamines. Preferably, the water-soluble polymeric blocks are made from poly(ethylene oxide). Preferably, at least 50% of the macromers are formed of synthetic materials (e.g., at least about 55%, at least about 60%, at least about 65%, at least about 70%, or at least about 75%).

The hydrophilic groups of the macromers may also be derived from natural materials. Useful natural and modified natural materials include carboxymethyl cellulose, hydroxyalkylated celluloses such as hydroxyethyl cellulose and methylhydroxypropyl cellulose, polypeptides, polynucleotides, polysaccharides or carbohydrates such as Ficoll™ polysucrose, hyaluronic acid and its derivatives, dextran, heparan sulfate, chondroitin sulfate, heparin, or alginate, and proteins such as gelatin, collagen, albumin, or ovalbumin. Preferably the percentage of natural material does not exceed about 50% percent.

The monomers are preferably covalently reactive, and thus form a covalently crosslinked gel. The crosslinked gels are elastic, and further are both elastic and compliant with soft tissue at low polymer concentrations.

In the preferred embodiment, the hydrogel is a “FocalGel™” or “FocalSeal™”, i.e., a biodegradable, polymerizable macromer having a solubility of at least about 1 g/100 ml in an aqueous solution comprising at least one water soluble region, at least one degradable region which is hydrolyzable under in vivo conditions, and free radical polymerizable end groups having the capacity to form additional covalent bonds resulting in macromer interlinking, wherein the polymerizable end groups are separated from each other by at least one degradable region. Exemplary FocalGel™ and FocalSeal™ compositions and hydrogels are described in U.S. Pat. No. 5,410,016 and U.S. Pat. No. 6,083,524, both of which incorporated herein by reference in its entirety. FocalGel™ and FocalSeal™ are available from Genzyme Corporation and are provided in a plurality of grades including S, L, and M.

In some embodiments, one or more commercially available FocalSeal products is blended with another (e.g., FocalSeal-L blended with FocalSeal-S) to provide a desired mix of properties (e.g., half life and stiffness) The individual polymeric blocks can be arranged to form different types of block copolymers, including di-block, tri-block, and multi-block copolymers. The most preferred embodiment is a di-block copolymer including a water-soluble block linked to a biodegradable block, with both ends capped with a polymerizable group, where the biodegradable blocks are a carbonate or hydroxyacid monomer such as a lactide monomer or oligomer.

Some of these structures described herein are depicted below. PEG, lactate and acrylate units are used solely for purposes of illustration.

### Some Basic Structures:

(CH₂—CH₂—O)ₓ=PEG repeat unit=(PEG)ₓ

(CO—(CH₂)₃—O)_{y} or (O—(CH₂)₃—CO)_{y}(depending on direction)=TMC repeat unit=(TMC)_{y}

(CO—CH(CH₃)—O)_{z} or (O—CH(CH₃)—CO)_{z}(depending on direction)=Lactate repeat unit=(LA)_{z}

—CO—CH═CH₂=Acrylate end group=AA

### Segmented PEG/TMC Copolymer:

HO—(O—(CH₂)₃—O—CO—)O_{y}—[(CH₂—CH₂—O)ₓ—(CO—O—(CH₂)₃—O)_{y}]ₙ—H or HO-(TMC)_{y}—[(PEG)ₓ-(TMC)_{y}]ₙ—H

### Segmented PEG/TMC/Lactate Terpolymer:

H—(O—CH(CH₃)—CO)_{z}—O—(O—(CH₂)₃—O—CO—)_{y}—[(CH₂—CH₂—O)ₓ—(CO—O—(CH₂)₃—O)_{y}]ₙ—(CO—CH(CH₃)—O)_{z}—H or HO-(LA)_{z}-(TMC)_{y}—[(PEG)ₓ-(TMC)_{y}]ₙ-(LA)_{z}-H

Segmented PEG/TMC Macromer (acrylated):

CH₂═CH—CO—(O—(CH₂)₃—O—CO—O—)_{y}[(CH₂—CH₂—O)ₓ—(CO—(CH₂)₃—O)_{y}]ₙ—CO—CH═CH₂ or AA-(TMC)_{y}—[(PEG)ₓ-(TMC)_{y}]ₙ-AA

### Segmented PEG/TMC/Lactate Terpolymer Macromer (Acrylated):

AA-(LA)_{z}-(TMC)_{y}—[(PEGₓ-(TMC)_{y}]ₙ-(LA)_{z}-AA

The biodegradable region is preferably hydrolyzable under in vivo conditions. For example, hydrolyzable group may be polymers and oligomers of glycolide, lactide, paradioxamone .epsilon.-caprolactone, other .-hydroxy acids, and other biologically degradable oligomers or polymers that yield materials that are non-toxic or present as normal metabolites in the body. Preferred poly(.alpha.-hydroxy acid)s are poly(glycolic acid), poly(DL-lactic acid) and poly(L-lactic acid). Other useful materials include poly(amino acids), poly(anhydrides), poly(orthoesters), and poly(phosphoesters). Polylactones such as poly(.epsilon.-caprolactone), poly(.epsilon.-caprolactone), poly(.delta.-valerolactone) and poly(gamma-butyrolactone), for example, are also useful.

As used herein, a carbonate is a functional group with the structure —O—C(O)—O—. The carbonate starting material can be derived from a cyclic carbonate, such as trimethylene carbonate (TMC), or a linear carbonate, such as dimethylcarbonate (CH₃O—C(O)—OCH₃). After incorporation into the polymerizable macromer, the carbonate will be present at least in part as R—O—C(O)—O—R′, where R and R′ are component residues of the macromer. More preferred carbonates for incorporation into the macromer are the cyclic carbonates, which can react with hydroxy-terminated polymers without release of water. Suitable cyclic carbonates include ethylene carbonate (1,3-dioxolan-2-one), propylene carbonate (4-methyl-1,3-dioxolan-2-one), trimethylene carbonate (1,3-dioxan-2-one) and tetramethylene carbonate (1,3-dioxepan-2-one).

In the most preferred embodiments, the macromers contain between about 0.3% and 20% of carbonate residues per macromer molecule, more preferably, between about 0.5% and 15% carbonate residues, and most preferably, about 1% to 5% carbonate residues. In those embodiments where hydroxy acid residues are desired, the macromer contains between about 0.1 and 10 residues per residue of carbonate, more preferably between about 0.2 and 5, and most preferably one or more such residue per macromer. In this preferred embodiment, the macromer includes a core of a hydrophilic poly(ethyleneoxide) oligomer (a.k.a. poly(ethyleneglycol) or PEG) with a molecular weight between about 400 and 40,000 Da, most preferably 20,000 Da; an extension on both ends of the core which includes 1 to 10 carbonate residues and optionally between one and five hydroxyacid residues, preferably alpha-hydroxy acid residues, most preferably lactic acid residues; wherein the total of all residues in the extensions is sufficiently small to preserve water-solubility of the macromer, being typically less than about 20% of the weight of the macromer, more preferably 10% or less. The ends are capped with ethylenically-unsaturated (i.e., containing carbon-carbon double bonds) caps, with a preferred molecular weight between about 50 and 300 Da, most preferably acrylate groups having a molecular weight of 55 Da. These materials are described in U.S. Pat. No. 6,177,095 to Sawhney, et al. (incorporated herein by reference in its entirety). See also U.S. Pat. No. 5,900,245 to Sawhney, et al. (incorporated herein by reference in its entirety).

In some embodiments, a macromer can contain a specific biodegradable region, which can modify the time to degradation of the resulting polymer. For example, in some embodiments, a macromer containing a lactate moiety as biodegradable region and end group provides a resulting hydrogel with an estimated degradation time in vivo of from about 3 to about 4 months. In some embodiments, a macromer containing a trimethylene carbonate moiety as a biodegradable region provides a resulting hydrogel with an estimated degradation time in vivo of from about 6 to about 12 months. In some embodiments, a polymer containing a dioxanone moiety as a biodegradable region provides a resulting hydrogel with an estimated degradation time in vivo of from about 6 to about 12 months. In some embodiments, a polymer containing a caprolactone moiety as biodegradable region provides a resulting hydrogel with an estimated degradation time in vivo of from about 1 to about 2 years. In some embodiments, a macromer without a biodegradable region can provide a resulting hydrogel with an estimated degradation time in vivo of at least about 2 years.

In some embodiments, a composition described herein is blended with another agent, for example, an agent used for soft tissue augmentation and/or repair such as a gel of hyaluronic acid such as hylan B or hylastan e.g., crosslinked, or collagen.

Other compounds can be added to the macromer containing compositions, for example, a drug to manage pain, such as lidocaine, anti inflammatory drugs, steroids, chemo therapeutics, or Botulinum Toxin. Stabilizers which prevent premature polymerization can be included;

typically, these are quinones, hydroquinones, or hindered phenols.

### Methods of Polymerization of Macromer Containing Compositions

Any method of covalent polymerization is potentially useful in the formation of the gels. The reactive groups may include, without limitation, ethylenically unsaturated groups, isocyanates, hydroxyls and other urethane-forming groups, epoxides or oxiranes, sulfhydryls, succinimides, maleimides, amines, thiols, carboxylic acids and activated carboxylgroups, sulfonic acids and phosphate groups. Ethylenically unsaturated groups include acrylates and other unsaturated carboxylic acids, vinylic and allylic groups, cinnamates, and styrenes. Activated carboxyl groups include anhydrides, carbonylimidazoles, succinimides, carbonyl nitrophenols, thioesters, O-acyl ureas, and other conjugated carbonyls. In general, any reactive group that will covalently bond to a second and that can maintain fluidity when exposed to water for enough time to allow deposition and reaction is of use in making a suitable reactive macromer. Due to their excellent stability and slow reactivity in aqueous solutions, ethylenically unsaturated reactive groups are preferred.

In some embodiments, the polymerization reaction need not result in covalent bonds. A number of materials are known which can form gel structures by changing the ionic conditions of the medium (e.g. alginate) or by changing the temperature of the medium (e.g., agarose, certain poloxamers). Polysaccharides are typical of these materials. Gel-like structures can be formed from proteins, such as gelatin or fibrin. While it may be more difficult to get these materials to adhere strongly to tissue, they are potentially of use in the hydrogels described herein.

Hydrogel formation can be accelerated by inclusion of small (non-macromeric) polymerizable molecules that can assist in linking larger, polymeric macromers. These typically have molecular weights less than about 1000 Da, more preferably less than 500 Da. For free radical polymerization, any of the common ethylenically unsaturated molecules can be used. These include derivatives of acrylic and methacrylic acid, such as acrylamide, hydroxyethyl methacrylate (HEMA), and diacrylated or polyacrylated glycols and oligoglycols. Allyl groups (e.g., allyl glycidyl ether) and vinyl groups (e.g., N-vinyl caprolactam and N-vinyl pyrrolidone) are also of use. Other unsaturated compounds include cinnamic acid and its esters, and maleic, fumaric and itaconic acids and their derivatives. Similar small molecules can be used to accelerate electrophilic/nucleophilic reactions, such as small polyamines, polyols and polythiols, polyisocyanates, and polysuccimidates.

### Methods of Synthesizing Macromers

The macromers described herein can be synthesized using means well known to those of skill in the art. General synthetic methods are found in the literature, for example in U.S. Pat. No. 5,410,016 to Hubbell et al., U.S. Pat. No. 4,243,775 to Rosensaft et al., and U.S. Pat. No. 4,526,938 to Churchill et al. (incorporated herein by reference in their entirety). For example, a polyethylene glycol backbone can be reacted with trimethylene carbonate (TMC) or a similar carbonate to form a TMC-polyethylene glycol terpolymer. The TMC-PEG polymer may optionally be further derivatized with additional degradable groups, such as lactate groups. The terminal hydroxyl groups can then be reacted with acryloyl chloride in the presence of a tertiary amine to end-cap the polymer with acrylate end-groups. Similar coupling chemistry can be employed for macromers containing other water-soluble blocks, biodegradable blocks, and polymerizable groups, particularly those containing hydroxyl groups.

When polyethylene glycol is reacted with TMC and a cyclic ester of a hydroxy acid such as glycolide or lactide. (This class of monomer is referred to as “lactides”), the reaction can be either simultaneous or sequential. The simultaneous reaction will produce an at least partially random copolymer of the three components. Sequential addition of a lactide after reaction of the PEG with the TMC will tend to produce an inner copolymer of TMC and one or more PEGs, which will statistically contain more than one PEG residue linked by linkages derived from TMC, with hydroxy acid moieting largely at the ends of the (TMC, PEG) region. Upon reaction of, for example, trimethylene carbonate (TMC) with polyethylene glycol (PEG), the TMC linkages in the resulting copolymers have been shown to form end linked species of PEG, resulting in segmented copolymers, i.e. PEG units coupled by one or more adjacent TMC linkages. The length of the TMC segments can vary. Coupling may also be accomplished via the carbonate subunit of TMC. These segmented PEG/TMC copolymers form as a result of transesterification reactions involving the carbonate linkages of the TMC segments during the TMC polymerization process when a PEG diol is used as an initiator. If the product of this first reaction step is then reacted with a reactive end-capping material, such as acryloyl chloride, a significant percentage of the macromer end groups can be PEG hydroxyls, resulting in the attachment of the reactive groups directly to one end of a non-biodegradable PEG molecule. Such a reaction of the PEG/TMC segmented copolymers can be prevented by adding additional segments of other hydrolyzable co-monomers (e.g. lactate, glycolate, 1,4-dioxanone, dioxepanone, caprolactone) on either end of the PEG/TMC segmented copolymer. The basic PEG/TMC segmented copolymer or the further reacted PEG/TMC/comonomer segmented terpolymer is then further reacted to form crosslinkable macromers by affixing reactive end groups (such as acrylates) to provide a macromer with reactive functionality. Subsequent reaction of the end groups in an aqueous environment results in a bioabsorbable hydrogel.

Polymerization is initiated by any convenient reaction, including photopolymerization, chemical or thermal free-radical polymerization, redox reactions, cationic polymerization, and chemical reaction of active groups (such as isocyanates, for example.) Polymerization is preferably initiated using photoinitiators. Photoinitiators that generate a free radical on exposure to light are well known to those of skill in the art. Free-radicals can also be formed in a relatively mild manner from photon absorption of certain dyes and chemical compounds. The polymerizable groups are preferably polymerizable by free radical polymerization. The preferred polymerizable groups are acrylates, diacrylates, oligoacrylates, methacrylates, dimethacrylates, oligomethacrylates, cinnamates, dicinnamates, oligocinnamates, and other biologically acceptable photopolymerizable groups.

These groups can be polymerized using photoinitiators that generate free radicals upon exposure to light, including UV (ultraviolet) and IR (infrared) light, preferably long-wavelength ultraviolet light (LWUV) or visible light. LWUV and visible light are preferred because they cause less damage to tissue and other biological materials than short-wave UV light. Useful photoinitiators are those which can be used to initiate polymerization of the macromers without cytotoxicity and within a short time frame, minutes at most and most preferably seconds. Exposure of dyes, preferably in combination with co-catalysts such as amine, to light, preferably visible or LWUV light, can generate free radicals. Light absorption by the dye causes the dye to assume a triplet state, and the triplet state subsequently reacts with the amine to form a free radical which initiates polymerization, either directly or via a suitable electron transfer reagent or co-catalyst, such as an amine. Polymerization can be initiated by irradiation with light at a wavelength of between about 200-1200 nm, most preferably in the long wavelength ultraviolet range or visible range, 320 nm or higher, and most preferably between about 365 and 550 nm.

Numerous dyes can be used for photopolymerization. Suitable dyes are well known to those of skill in the art. Preferred dyes include erythrosin, phloxime, rose bengal, thionine, camphorquinone, ethyl eosin, eosin, methylene blue, riboflavin, 2,2-dimethyl-2-phenylacetophenone, 2-methoxy-2-phenylacetophenone, 2,2-dimethoxy-2-phenyl acetophenone, other acetophenone derivatives, and camphorquinone. Suitable co-initiators include amines such as N-methyl diethanolamine, N,N-dimethyl benzylamine, triethanol amine, triethylamine, dibenzyl amine, N-benzylethanolamine, N-isopropyl benzylamine. Triethanolamine is a preferred co-initiator.

Suitable chemical, thermal and redox systems may initiate the polymerization of unsaturated groups by generation of free radicals in the initiator molecules, followed by transfer of these free radicals to the unsaturated groups to initiate a chain reaction. Peroxides and other peroxygen compounds are well known in this regard, and may be considered as chemical or thermal initiators. Azobisbutyronitrile is a chemical initiator. A combination of a transition metal, especially iron, with a peroxygen and preferably a stabilizing agent such as glucuronic acid allows generation of free radicals to initiate polymerization by a cycling redox reaction.

It is also possible to use the macromers with other types of linking reactions. For example, a macromer could be constructed with amine termination, with the amine considered as an active group; and another macromer could be constructed with isocyanate termination, with the isocyanate as the active group. On mixing, the materials will spontaneously react to form a gel. Alternatively, an isocyanate-terminated macromer could be polymerized and crosslinked with a mixture of diamines and triamines. Other pairs of reactants include maleimides with amines or sulfhydryls, or oxiranes with amines, sulfhydryls or hydroxyls or n-hydroxysuccinimide with amines, or sulfhydryls.

### Physical and Chemical Properties of Macromers and Hydrogels

The copolymers and macromers described herein generally have tailorable properties such as solubility and solution viscosity properties. The hydrogels can have tailorable physical properties, such as modulus, elasticity, and degradation rate.

For a given solution concentration in water, the viscosity is generally affected by the degree of end linking, the length of the TMC (and other hydrophobic species) segments, and the molecular weight of the starting hydrophilic polymers (e.g., PEG). The modulus of the hydrogel is affected by the molecular weight between crosslinks. The hydrogel degradation rate can be modified, for example, by adding a second, more easily hydrolyzed comonomer (e.g. lactate, glycolate, 1,4-dioxanone) as a segment on the ends of the basic (PEG/TMC) copolymer prior to adding the crosslinkable end group to form the macromer.

In some cases it is desirable to increase the viscosity of the macromer solution at the time of application to the tissue so that the macromer remains more firmly at the site of application. Polymers which can be used to increase the viscosity of the macromer solution include: glycosaminoglycans (GAG) such as hyaluronic acid (HA), carboxymethyl cellulose (CMC), dextran, dextran sulfate, and polyvinylpyrrolidone (PVP). These are typically added to the macromer solution immediately before application to the tissue.

The length of time it takes for the hydrogel to biodegrade may be tailored to provide a hydrogel that remains in the soft tissue for at least about 2 weeks, e.g., at least about 1 month, at least about 2 months, at least about 3 months, at least about 4 months, at least about 5 months, at least about 6 months, at least about 8 months, at least about 7 months, at least about 9 months, at least about 12 months, at least about 15 months, at least about 18 months, at least about 21 months, or at least about 24 months.

### Compliance Properties

The hydrogels are preferably highly compliant with the tissue in to which they are injected. Thus, the hydrogels stretch and bend along with the tissue. It is preferable that the response to stress within the limits of general use of the soft tissue be substantially elastic, i.e., reversible. Thus the hydrogel should remain as a coherent material one implanted.

The compliance properties of the material herein described are those of the material after it has polymerized to form a polymerized material such as a hydrogel described herein. As used herein, “polymerized material” includes material which forms by or covalent reaction of monomer precursor molecules, including for example, a hydrogel described herein. Preferably, the polymerized material is formed by covalent reactions of the monomers.

It can be very difficult to measure the elastic properties of the material upon application (e.g., when adhered to tissue). The mechanical properties can therefore be measured on samples made in vitro, either in a mold, or, as in the lap-shear test, in contact with standardized tissue. Such measurements must be corrected to conditions applicable to tissue treatment, including the diluting effects of polymerization reagents, or of fluids on the tissue. Thus, a filler solution may be injected in to tissue at a concentration of 30%, but it may be diluted to 15% effective concentration by dilution with blood or plasma. Similarly, especially in the case of fibrin sealant, the polymer concentration may be reduced by mixing with polymerizing reagents. Where appropriate, such corrections have been taken into account in the descriptions herein. Materials may be equilibrated with water before testing either by absorption or syneresis.

In light of these observations, an effective material for forming a compliant hydrogel, for example to augment and or repair soft tissue, has a strain or elongation before fracture substantially similar to or at least as great as the expected strain during normal use of the tissue (e.g., soft tissue) in to which it is injected, and the elongation of the polymerized material is preferably reversible. This is to avoid either detachment from the surrounding tissue or fracture, or limitation of the tissue's natural expansion. Preferably, the effective compliant material will have a reversible elongation at least about 150% as great, more preferably at least about 200% as great, and still more preferably at least about 300% as great as the expected strain of the tissue.

The polymerized material thus may be designed and selected for application to different tissue (e.g., soft tissue), to have an elongation at rupture which is similar to or greater than the elongation of the tissue in vivo during its function. The elongation at rupture of the polymerized material can be, for example, greater than 100% or 200%, or optionally greater than 300% or 400%. In some embodiments, the elongation at rupture of the polymerized material may be between for example 100% and 700%, depending on the tissue properties. In some applications, an elongation at rupture greater than 700% is useful. This property can be varied, for example, to be optimized specific to the soft tissue being augmented.

In addition, the compliant material, for example in applications to augment and or repair soft tissue, preferably should have a normalized compliance that is comparable in magnitude to the normalized compliance of the tissue to which it is applied. The material will be operative even when the material's normalized compliance is much greater than the normalized compliance of the tissue.

In cases where minimal modification of the natural expansion and contraction of a tissue is desired, the preferred range of the normalized compliance ratio extends from about 0.05 to about 3, preferably from about 0.1 to about 2.0, and more preferably from about 0.1 to about 1.0. In some cases, for example when the tissue is soft tissue, a value of the elastic modulus of less than about 150 kPa, preferably less than 100 kPa, more preferably less than about 50 kPa, and most preferably less than about 30 kPa is preferred.

To obtain the desired ratio of the normalized compliance of the polymerized material to the normalized compliance of tissue, the overall force required to stretch the hydrogel layer should be adjusted, since that of the tissue is fixed. The adjustment can be accomplished by any of several known methods, including the alteration of the thickness of the layer of the polymerized material (e.g., hydrogel), or the variation of the polymer concentration, or of the polymer crosslink density, or of other properties of the material. The properties of the precursor materials and the reaction conditions may be adjusted to produce desired other properties of the polymerized material.

Where prevention of tissue deformation is desired, for example during a healing period, the parameters of the tissue filler can be adjusted so that the normalized compliance ratio is significantly in excess of 1.

In many applications, such as augmenting and/or repairing soft tissue, the viscosity of the precursor materials can be tailored to obtain optimal filler materials. Higher viscosities can favor retention of the uncured or unpolymerized filler at the site of injection, and minimize displacement of the filler by the presence of bodily fluids in the tissue. However, higher viscosities make the material more difficult to inject. A suitable range of viscosity, for example augmenting and/or repairing soft tissue is in the range of about 200 cP (centipoise) to about 40,000, preferably about 500 to about 5000 cP, and more preferably about 700 to about 1200 cP. The optimal viscosity will depend on the site of application and the nature of the condition which is to be alleviated by the application of the material.

In a preferred embodiment, the hydrogel composition is selected to provide acceptable levels of fibrosis or tissue reaction, for example a mild level of fibrosis. This can be achieved through the selection of the reactive formulation, and other techniques known to those skilled in the art in drug delivery utilizing polymeric delivery devices. A mild fibrotic response to the hydrogel, resulting in mild fibrosis can potentially extend the functional life of the hydrogel, providing matrix material from the subject in the area of the hydrogel material.

### Methods of Use

Surgical applications for an injectable, biodegradable macromer containing composition and resulting hydrogel include, but are not limited to: facial contouring (frown or glabellar line, acne scars, cheek depressions, vertical or perioral lip lines, marionette lines or oral commissures, worry or forehead lines, crow's feet or periorbital lines, deep smile lines or nasolabial folds, smile lines, facial scars, lips and the like); periurethral injection including injection into the submucosa of the urethra along the urethra, at or around the urethral-bladder junction to the external sphincter; uretheral injection for the prevention of urinary reflux; injection into the tissues of the gastrointestinal tract for the bulking of tissue to prevent reflux; to aid in sphincter muscle coaptation, internal or external, and for coaptation of an enlarged lumen; injection into anatomical ducts to temporarily plug the outlet to prevent reflux or infection propagation; larynx rehabilitation after surgery or atrophy; lumpectomy filler, and any other soft tissue which can be augmented for cosmetic or therapeutic effect.

Surgical specialists could use a composition or hydrogel described herein, including but are not limited to, plastic and reconstructive surgeons; dermatologists; facial plastic surgeons, cosmetic surgeons, otolaryngologists; urologists; gynecologists; gastroenterologists; ophthalmologists; and any other physician qualified to utilize such a product.

Additionally, to facilitate the administration and treatment of patients with compositions and hydrogels described herein, pharmaceutically active compounds or adjuvants can be administered therewith. Pharmaceutically active agents that may be coadministered with the compositions and hydrogels include but are not limited to anesthetics (such as lidocaine) and antiinflammatories (such as cortisone or non-steroidal). Thus, the compositions may further comprise a drug such as a non-steroidal anti-inflammatory, an analgesic, a vitamin such as E, C, A, D or K, an anti-oxidant, an alpha hydroxyl acid such as lactic acid or a polymer capable of releasing such drug, vitamin, anti oxidant or alpha-hydroxyacid or any combination thereof.

Exemplary non-steroidal anti-inflammatories may be selected from those identified in The Merk Index and include, but are not limited to, aspirin, ibuprofen, indomethacin, ketoprofen, naproxen, niflumic acid, prioxicam, diclofenac, tolmetin, fenoclofenac, meclofenamate, mefenamic acid, etodolac, sulindac, carprofen, fenbufen, fenoprofen, flurbiprofen, ketoprofen, oxaprozin, tiaprofenic acid, phenylbutazone diflunisal, or salsalate, and salts and analogues thereof.

Exemplary anesthetics may be selected from those identified in The Merk Index and include, but are not limited to, benzocaine, bupivacaine, lidocaine, mepivacaine, prilocaine, orpropoxycaine and salts and analogues thereof.

Exemplary anti-oxidant may be selected from, but are not limited to, vitamin E, vitamin C, ascorbyl palmitate, benzoic acid, benzyl hydroxybenzoate, bronopol, butyl hydroxybenzoate, butylated hydroxyanisole, butylated hydroxytoluene, chlorbutol, cinnamic acid, dehydroacetic acid, diethyl pyrocarbonate, ethoxyquin, ethyl hydroxybenzoate, isoascorbic acid, methyl hydroxybenzoate, monothioglycerol, nordihydroguairetic acid, phenethyl alcohol, phenoxyethanol, Q-phenylphenol, potassium sorbate, propyl hydroxybenzoate, sodium benzoate, sodium butyl hydroxybenzoate, sodium dehydroacetate, sodium diacetate, sodium ethyl hydroxybenzoate, sodium isoascorbate, sodium methyl hydroxybenzoate, sodium Q-phenylphenol, sodium propyl hydroxybenzoate, sorbic acid, or thiodipropionic acid and salts or derivatives thereof.

The compositions can be administered with a syringe and needle or a variety of devices. Several delivery devices have been developed and described in the art to administer viscous liquids such as the carpal devices described by Dr. Orentriech in U.S. Pat. Nos. 4,664,655 and 4,758,234 which are hereby incorporated by reference. Additionally, to make delivery of the compositions as easy as possible for the doctors, a leveraged injection ratchet mechanism or powered delivery mechanism may be used. It is currently preferred for the compositions to be preloaded in a cylindrical container or cartridge having two ends. The first end would be adapted to receive a plunger and would have a movable seal placed therein. The second end or outlet would be covered by a removable seal and be adapted to fit into a needle housing to allow the compositions in the container to exit the outlet and enter a needle or other hollow tubular member of the administration device. It is also envisioned that the compositions could be sold in the form of a kit comprising a device containing the composition. The device having an outlet for said composition, an ejector for expelling the composition and a hollow tubular member fitted to the outlet for administering the composition into an animal.

Once the composition is administered to the subject, the composition is polymerized, for example, by irradiating through the skin of the subject. The subject can be subjected to a transilluminating light, which penetrates the skin and initiates polymerization of the administered composition. When polymerization is achieved using radiation, the subject is generally administered radiation by illumination for at least about 10 seconds, e.g., at least about 15 seconds, at least about 20 seconds, at least about 25 seconds, at least about 30 seconds, at least about 35 seconds, at least about 45 seconds, at least about 60 seconds, at least about 90 seconds, or at least about 2 minutes.

The composition can be shaped simultaneously with the polymerization of the composition into a hydrogel. For example, a doctor or surgeon can manipulate the shape of the composition while polymerizing the composition (e.g., via radiation) to thereby provide a desired shape of the resulting hydrogel. In some embodiments, the composition is shaped mechanically by the doctor or surgeon, using his hand or a tool or mold to provide the desired shape. In some embodiments, the composition is injected into a cavity in the subject, thereby primarily taking the shape of the cavity when polymerized to become a hydrogel.

In some embodiments, a composition is administered to a subject in an iterative manner, such that at least two, for example, 3, 4, or 5 applications of the composition are provided to the subject, where the composition is polymerized between each new administration of the composition. The iterative application process can provide improved control of the final shape of the hydrogel, allowing a more customized look for the subject.

In some embodiments, a composition is administered to a subject with a chemical initiation system or a two component system such as isocyanate/amine, and can be formulated to give a “working time” to allow injection and shaping.

### Packaging

The compositions described herein can be packaged in any convenient way, and may form a kit including for example separate containers, alone or together with the application device. The reactive monomers are preferably stored separately from the initiator, unless they are co-lyophilized and stored in the dark such as in a red tinted syringe, or otherwise maintained unreactive. Dilute initiator can be in the reconstitution fluid; stabilizers are in the macromer or syringe; and other ingredients may be in either vial, depending on chemical compatibility. If a drug is to be delivered in the composition, it may be in any of the vials, or in a separate container, depending on its stability and storage requirements.

A number of embodiments of the invention have been described. Nevertheless, it will be understood that various modifications may be made without departing from the spirit and scope of the invention. Accordingly, other embodiments are within the scope of the following claims.

What is claimed is:

1. A method of repairing or augmenting soft tissue in a subject, the method comprising
a. injecting into a subject in need thereof a composition comprising a biodegradable, polymerizable macromer, the macromer comprising a water soluble polymer modified with one or more biodegradable moieties; and
b. polymerizing the macromer to provide a hydrogel wherein the hydrogel to soft tissue have a normalized compliance ratio of from about 0.05 to about 3, thus repairing or augmenting the soft tissue.

2. The method of claim 1, wherein the compliance ratio is from about 0.1 to about 2.0 relative to the soft tissue.

3. The method of claim 2, wherein the compliance ratio is from about 0.1 to about 1.0 relative to the soft tissue.

4. The method of claim 1, wherein the macromer is polymerized by irradiating through the skin of the subject with visible light.

5. The method of claim 1, wherein the subject is irradiated with visible light for from about 10 seconds to about 120 seconds.

6. The method of claim 5, wherein the subject is irradiated with visible light for at least about 30 seconds.

7. The method of claim 6, wherein the subject is irradiated with visible light for at least about 40 seconds.

8. The method of claim 1, wherein the macromer is polymerized by irradiating the subject with blue-green light.

9. The method of claim 1, wherein the macromer is polymerized by irradiating the subject with thermal energy.

10. The method of claim 1, wherein the water soluble polymer is PEG.

11. The method of claim 10, wherein the PEG has a molecular weight of from about 10,000 to about 35,000 Daltons.

12. The method of claim 1, wherein the water soluble polymer is a block-copolymer.

13. The method of claim 12, wherein the block-copolymer is an ethyleneoxide and propyleneoxide.

14. The method of claim 1, wherein the macromer is biodegradable.

15. The method of claim 1, wherein the macromer comprises a plurality of hydrolysable linkages.

16. The method of claim 15, wherein the hydrolyzable linkages are selected from the group consisting of esters or carbonates.

17. The method of claim 1, wherein the water soluble polymer is modified with an acrylate-capped poly (L-lactide).

18. The method of claim 17, wherein the water soluble polymer is PEG.

19. The method of claim 1, wherein the water soluble polymer is modified with a poly (trimethylene carbonate).

20. The method of claim 19, wherein the water soluble polymer is PEG.

21. The method of claim 1, wherein the water soluble polymer is modified with an poly (L-lactide) and poly (trimethylene carbonate) and an acrylate endcap.

22. The method of claim 21, wherein the water soluble polymer is PEG.

23. The method of claim 1, wherein the composition further comprises a photo-initiator.

24. The method of claim 23, wherein the photoinitiator is a dye.

25. The method of claim 24, wherein the dye is eosin.

26. The method of claim 1, wherein the composition further comprises a rheology modifier.

27. The method of claim 26, wherein the rheology modifier is HA or CMC.

28. The method of claim 1, wherein the composition is substantially free of organic solvent.

29. The method of claim 1, wherein the hydrogel has a strain or elongation before fracture substantially similar to the expected strain during normal use of the soft tissue to which it augments or repairs.

30. The method of claim 1, wherein the hydrogel has a strain or elongation before fracture greater than the expected strain during normal use of the soft tissue to which it augments or repairs.

31. The method of claim 1, wherein the hydrogel has a reversible elongation at least about 150% as great as an expected strain of the soft tissue which is augments or repairs.

32. The method of claim 1, wherein the hydrogel has an elastic modulus which is less than about 150 kPa.

33. The method of claim 1, wherein the hydrogen has an ultimate yield stress of from about 500 to about 2,000 psi.

34. The method of claim 1, wherein the macromer is injected subdermally.

35. The method of claim 34, wherein the macromer is polymerized by irradiating least a part of the skin of the subject.

36. The method of claim 35, wherein the skin is irradiated for at least about 30 seconds.

37. The method of claim 1, wherein the macromer is injected intradermally.

38. The method of claim 37, wherein the macromer is polymerized by irradiating at least a part of the skin of the subject.

39. The method of claim 38, wherein the skin is irradiated for at least about 30 seconds.

40. The method of claim 1, further comprising shaping the macromer.

41. The method of claim 40, wherein the macromer is shaped during polymerization of the macromer.

42. The method of claim 41, wherein the macromer is polymerized by irradiating through the skin of the subject.

43. The method of claim 1, comprising repeating steps a) and b) of claim 1 at least one time.

44. The method of claim 1, comprising repeating steps a) and b) of claim 1 at least two times.

45. The method of claim 1, wherein the subject is a mammal.

46. The method of claim 45, wherein the subject is a human.

47. The method of claim 1, the method comprising repairing facial tissue.

48. The method of claim 47, the method comprising decreasing the appearance of at least one facial line, wrinkle, crease, or fold.

49. The method of claim 1, the method comprising augmenting breast, lip, cheek, chin, forehead, buttocks, hand, neck or earlobe tissue in a subject.

50. The method of claim 1, the method comprising decreasing the appearance of a dermal dimple.

51. The method of claim 50, wherein the dimple is a component of a scar.

52. The method of claim 1, wherein the composition is administered with a red tinted syringe.

53. The method of claim 1, wherein the soft tissue remains substantially augmented or repaired for at least about 1 month.

54. The method of claim 53, wherein the soft tissue remains substantially augmented or repaired for at least about 2 months.

55. The method of claim 54, wherein the soft tissue remains substantially augmented or repaired for at least about 6 months.

56. The method of claim 1, wherein the hydrogel elicits a mild fibrotic response in the subject.

57. The method of claim 1, wherein the composition comprises a two part system, and wherein the polymerization is initiated via a redox system.

58. The method of claim 57, wherein the polymerization occurs over a period of from about 30 seconds to about 2 minutes.

59. The method of claim 1, wherein the composition further comprises a drug such as an non-steroidal anti-inflammatory, an analgesic, a vitamin such as E, C, A, D or K, an anti-oxidant, an alpha hydroxyl acid such as lactic acid or a polymer capable of releasing such drug, vitamin, anti oxidant or alpha-hydroxyacid or any combination thereof.
